(19) United States
(12) Patent Application Publication
(10) Pub. No.: US 2009/0093669 A1
(43) Pub. Date: Apr. 9, 2009

(54) MAGNETOPHORESIS APPARATUS AND METHOD OF USE

(76) Inventors: WILLIAM A. FARONE, IRVINE, CA (US); W. BRADLEY WORTHINGTON, NASHVILLE, TN (US)

Correspondence Address: WYATT, TARRANT & COMBS, LLP, 1715 AARON BRENNER DRIVE, SUITE 800, MEMPHIS, TN, 38120-4367, US

(21) Appl. No.: 12/245,615

(22) Filed: Oct. 3, 2008

Related U.S. Application Data
(60) Provisional application No. 60/977,927, filed on Oct. 5, 2007.

(51) Int. Cl. A61N 2/08; A61N 1/30; A61K 31/16; A61K 31/245; A61K 31/445; A61K 31/167; A61K 31/381; A61K 31/192; A61K 31/485; A61K 31/407; A61K 31/137; A61P 43/00

(52) U.S. Cl. 600/15; 604/20; 514/625; 514/646; 514/330; 514/626; 514/447; 514/570; 514/282; 514/413

(57) ABSTRACT

A method and apparatus for administering drugs through a dermal or mucosal layer such that the treatment device may be applied to the mammal for the transdermal or transmucosal administration of various drugs, pharmaceuticals, salts and prodrugs thereof is taught herein. The magnetic device has at least one magnet to provide an effective magnetic sphere of influence of the magnetic field at the desired site of action. The tissue-permeable drug formulation is positioned on the site of action and the magnetic device is positioned adjacent to tissue-permeable drug formulation wherein the magnetic sphere of influence of the magnetic field moves the drug through the dermal or mucosal layer by interaction with the magnetic moment (induced or permanent) of the drug sufficient to increase the movement of the drug beyond the movement caused by diffusion alone.

### CROSS REFERENCE TO RELATED APPLICATIONS

This application claims priority of U.S. Provisional Application No. 60/977,927, filed Oct. 5, 2007, the contents of which are incorporated fully by reference.

### STATEMENT REGARDING SPONSORED RESEARCH OR DEVELOPMENT

Not applicable.

### REFERENCE TO A MICROFICHE APPENDIX

Not applicable.

### FIELD OF THE INVENTION

The present invention relates to a method of utilizing magnetic devices and electrical fields for transdermal delivery of drugs with therapeutic application for mammals and more particularly to the method of use of a static magnetic field including the use of a quadrapolar magnetic transdermal drug delivery device. The present invention further relates to methods of use and/or potentiation of pharmaceuticals and focusing and for concentrating the drug to the active site. All of the following inventions of the present inventors are incorporated in patent applications which disclose the use of magnetic arrays useful in the treatment of physiological conditions in a human and are thus relevant, and are incorporated by reference herein: U.S. application Ser. No. 11/469,346 filed on Aug. 31, 2006 [2008/0103350 A1], U.S. application Ser. No. 11/678,528, filed on Feb. 23, 2007 [20080107986].

### BACKGROUND OF THE INVENTION

Transdermal delivery of pharmaceuticals has become an accepted way of introducing drugs into mammals for those compounds for which diffusion through the dermal barrier occurs either alone or through the use of adjuvants to assist skin penetration. Recently, direct current (DC) electric fields have been used to accelerate the movement of the drug through the dermal barrier. This technology has been called iontophoresis. An iontophoresis device is defined in 21 CFR 890.5525 which states “An iontophoresis device is a device that is intended to use a direct current to introduce ions of soluble salts or other drugs into the body . . . ”. BlueCross BlueShield of Tennessee in their Medical Policy Manual in 2006 stated that iontophoresis as a transdermal drug delivery technique for medical indications is considered investigational. Various aspects of iontophoresis are described in the following patents and patent applications: iontophoretic drug delivery system with a controller for electric current in WO 96/10442; low cost electrodes for an iontophoretic device in EP 0 957 979 B1; an iontophoretic drug delivery device comprising a fluid reservoir and electrode assembly in U.S. Pat. No. 7,137,965 B2; a transdermal or transmucosal drug delivery device capable of reducing power consumption in U.S. Pat. No. 7,200,433 B2; an electrotransport of an anesthetic and vasoconstrictor in US 2007/0078372 A1 (WO 2007/041475 A2); a transdermal device system including one active agent reservoir in US 2007/0083186 A1 (WO 2007/041314 A2); a device with at least one active agent reservoir in US 2007/110810 A1 [WO 2007/041119 A1]; a device for abrading the stratum corneum of the skin to increase transdermal delivery in US 2006/0264893. Iontophoresis employs an electromotive/electrical force and/or current to transfer the active agent to the skin interface.

Molecules are made up of atoms wherein the nucleus is positively charged and the atoms are surrounded by swarms of the negatively charged electrons. Complicated molecules, like drugs, are systems of atoms held together by very complicated interactions of the electrons. There are only a few atoms, like helium and neon (so called “inert gases”) where the distribution of electrons is such that they do not form many “bonds” with other atoms. When atoms are put together into molecules, it is often the case that the charges will not be uniform in space and the spatial arrangement results in a partial charge separation leading to a property known as the “dipole moment”.

An electric field moves particles in a solvent if those particles have a charge, either full charges like “ions” or partial charges like those molecules with a dipole moment. The speed with which the molecules will move turns out to be directly proportional to the square of the dipole moment. It also turns out that the speed is directly proportional to the magnitude of the electric field and inversely proportional to the viscosity of the solvent and to the square of the size of the molecule. The electric field imparts movement to the molecule through a force exerted by the field itself sometimes referred to as “radiation pressure”. This movement is primarily longitudinal (in the direction of the field) and is in addition to the natural tendency for the molecule to diffuse due to a concentration gradient.

Transdermal administration of drugs has advantages over other means of administration such as ease in administering, maintaining levels of drugs in the blood, avoidance of gastrointestinal drug reactions, and avoidance of poor gastrointestinal absorption. Electric iontophoresis requires a source of external power to create the electric field to stimulate the movement of the compound through the dermal layer or mucous membrane. Because transdermal administration is highly desirable, there is a need for an alternative method that does not require an external electric current.

### BRIEF SUMMARY OF THE INVENTION

A method and apparatus for administering drugs through a dermal layer such that the treatment device may be applied to the mammal for the transdermal administration of various drugs, pharmaceuticals, salts and prodrugs thereof is taught herein. The method and apparatus include a static magnetic device which has at least one magnet to provide an effective magnetic sphere of influence of the magnetic field at the desired site of action. The method and apparatus may include an electromagnetic (TENS) device coupled with the static magnetic device. The static magnetic field is independent of the arrangement used to produce the electro-magnetic field. The field directions and strengths that cause the molecules to be moved by the field move by diffusion are defined herein. In exemplary implementations the tissue-permeable drug formulation is positioned on the site of action and the magnetic device is positioned adjacent to tissue-permeable drug formulation wherein the magnetic sphere of influence of the magnetic field moves the drug through the dermal layer by interaction with the magnetic moment (induced or permanent) of the drug sufficient to increase the movement of the drug beyond the movement caused by diffusion alone.

In a further implementation the magnetic field is at least 1.5 mT at the desired depth of penetration of the drug.

In another implementation iontophoresis is used simultaneously or separately as an enhancer.

In a further implementation the drug formulation contains pain-relieving compounds, their pharmaceutically acceptable salts or prodrugs. The pain-relieving compounds may be selected from the group consisting of capsaicin, ketamine, bupivacaine, lidocaine, prilocaine, xylocaine, articaine, chloroprocaine, mepivacaine, procaine, opiates, ketoprofen, codeine, ephedrine, epinephrine, and ketorolac. In an additional implementation other active pharmaceutical ingredients such as insulin may be contained in the drug formulation. In further implementations, the drug formulations may contain additional compounds that are selected from preservatives, stabilizers, creams, gels, excipients, and isotonic solvents.

The invention includes a method for administering drugs through a dermal layer or mucous membrane such that the treatment apparatus may be applied to the mammal for the transdermal or transmucosal administration of various drugs, pharmaceuticals, salts and prodrugs thereof. The-apparatus provides a magnet array of alternating polarity in which the magnetic poles are separated by a predetermined distance to provide an effective magnetic sphere of influence of the magnetic field at the desired site of action from the magnetic fields of all adjacent poles. In a further implantation the tissue permeable drug formulation is positioned on the mucous membrane or dermal layer at the site of action and the magnetic apparatus is positioned adjacent to the drug formulation such that the magnetic sphere of influence of the magnetic field moves the drug into and through the mucous or dermal layer.

In a further implementation the treatment device provides a magnet array of two magnets. In yet another implementation the treatment device provides a magnet array of four magnets.

In another implementation the plurality of magnets are so positioned that positive and negative poles are aligned in different planes or away from one another with sufficient strength to achieve the desired effective magnetic field. Yet another implementation the plurality of magnets are arranged in alternating polarity with a flux focusing medium surrounding the static magnetic poles.

In a further implementation each of the magnets has a residual induction value of at least about 1,400 to 1,500 mT and each of the magnets has a diameter of about 12.7 mm and a thickness of about 6.35 mm. In yet another implementation the magnetic field at the center of the North Pole of the magnets is about 530 mT. The magnetic field gradient at the site of action of adjacent poles is about at least 1.5 mT/mm in the direction perpendicular to the axis from the magnetic poles.

In another implementation iontophoresis is used as an enhancer either simultaneously or sequentially with the magnetic apparatus. In a further implementation a transcutaneous electrical nerve stimulator (TENS) unit using pulsed DC is provided such that the magnetic array and the TENS unit simultaneously accelerate the diffusion of the pharmaceutical formulation dermally.

Definitions:

In accordance with the present invention and as used herein, the following terms are defined with the following meanings, unless explicitly stated otherwise.

The term “mammal” refers to any of various warm-blooded vertebrate animals including those in the class Mammalia, and includes humans.

The term “Tesla” refers to a unit of magnetic flux density or magnetic field intensity in the International System of Units equal to the magnitude of the magnetic field vector necessary to produce the force of one Newton on a charge of one coulomb moving perpendicular to the direction of the magnetic field vector with a velocity of one meter per second.

The term “Gauss” refers to a unit of magnetic flux and is exactly 10 times smaller than the Tesla.

The term “residual induction value” refers to a property of magnetic materials also known as magnetization and is related to the strength of the field that a given material is capable of producing.

The term “gradient” refers to the rate at which a physical quantity, such as temperature or pressure, increases or decreases relative to change in a given variable, especially distance. The term may also refer to a vector having coordinate components that are the partial derivatives of a function with respect to its variables.

The terms, “fat free” or “low fat”, refers to beef tissue cells that contain 0.5 gms of fat or less (1%) per 21 CFR 101.62(b).

The term “diffusion coefficient” refers to the movement of a material in a direction as described by Equation 1.

The term “skin” refers the external covering of a mammal body. It contains two layers: a thin outer layer, the epidermis and a thicker inner layer, the dermis. It is also refers to the stratum corneum covered skin and mucosal membranes.

The term “drug” refers to any pharmaceutically active compound, its pharmaceutically acceptable salts and prodrugs thereof, including but not limited to compounds that treat diseases, injuries, pain, undesirable symptoms, and improve or maintain health.

The term “TENS” refers to a transcutaneous electrical nerve stimulator which is a device that produces pulsed electrical currents that are used to stimulate nerves primarily for the relief of pain.

The term “mucous membrane: refers to a membrane lining all body passages that communicate with the air, such as the respiratory and alimentary tracts, and having cells and associated glands that secrete mucus.

The abbreviation “QMA” refers to Quadrapolar Magnetic Array.

### BRIEF DESCRIPTION OF THE DRAWINGS

The foregoing aspects and advantages of present disclosure will become more readily apparent and understood with reference to the following detailed description, when taken in conjunction with the accompanying drawings, wherein:

FIG. 1 illustrates a skin model for drug penetration according to the invention.

FIG. 2 is a series of curves illustrative of various times for an exemplary diffusion of a drug according to the invention.

FIG. 3 is a series of curves illustrating the same drug diffusion using a TENS apparatus.

FIG. 4 illustrates the control test diffusion of capsaicin in low fat beef cells.

FIG. 5 illustrates the diffusion of capsaicin in low fat beef cells with a magnetic field applied.

FIG. 6 illustrates the diffusion of capsaicin in low fat beef cells with an applied TENS electric field.

FIG. 7 illustrates the control test diffusion of capsaicin in 15% fat beef cells.

FIG. 8 illustrates the diffusion of capsaicin in 15% fat beef cells with a magnetic field applied.

FIG. 9 illustrates the diffusion of capsaicin in 15% fat beef cells with an applied TENS electric field.

FIG. 10 illustrates the control test diffusion of lidocaine in low fat beef cells.

FIG. 11 illustrates the diffusion of lidocaine in low fat beef cells with a magnetic field applied.

FIG. 12 illustrates the diffusion of lidocaine in low fat beef cells with an applied TENS electric field.

FIG. 13 illustrates a suitable magnet field with a distance profile.

FIG. 14 illustrates the field gradient at 6 mm above a quadrapolar array.

FIG. 15 illustrates the field gradient at 15 mm above a quadrapolar array.

FIG. 16 illustrates the control test diffusion of insulin in low fat beef cells

FIG. 17 illustrates the diffusion of insulin in low fat beef cells with a magnetic field applied.

FIG. 18 illustrates the diffusion of low fat beef cells with an applied TENS electric field.

FIG. 19 is a perspective view of a single permanent magnet on the drug formulation/diffusion substrate.

FIG. 20 is a perspective view of an embodiment composed of a configuration of two magnets located on the drug formulation substrate.

FIG. 21 is a perspective view of a quadrapolar magnetic array on the drug formulation substrate.

FIG. 22 illustrates an array of TENS electrodes and a single QMA according to the invention.

FIG. 23 is an illustration of the magnetic field effect on the diffusion of drug molecules into the subdermal tissue of a mammal.

The Figures are diagrammatic and are not drawn to scale. Corresponding parts generally bear the same reference numerals.

### DETAILED DESCRIPTION OF THE INVENTION

Before explaining the present invention in detail, it is to be understood that the disclosure herein is not to be limited to the particular implementations and that it may be practiced or carried out in various ways and various configurations.

Magnetic and Electrical Fields

By the present invention it was found that magnetic fields can be used, either singly or in combination with electric fields to control the penetration of drugs through the dermal surface. A method is taught wherein the field strength of the electric field is matched to the dipole moment and polarizability and/or the magnetic field is matched to the magnetic moment and magnetizability of the drug molecule. These method permits the use of virtually any drug for transdermal application.

These two fields behave in very different ways. The electric field imparts movement to the molecule through a force exerted by the field itself which is sometimes referred to as “radiation pressure”. This movement is primarily longitudinal (in the direction of the field) and is in addition to the natural tendency for the molecule to diffuse due to a concentration gradient. We have discovered that the static magnetic field, however, uses the movement of the drug molecule itself (which is undergoing natural diffusion) to accelerate the movement of the molecules both longitudinally (in the direction of the field) and laterally (transverse to the field based on the field gradient). The-effect of the lateral activity of the drug molecule facilitates its longitudinal diffusion into the tissue. While electric iontophoresis requires a source of external power to create the electrical field, the magnetic effect (magnetophoresis) requires no external power since the force comes from the natural movement of the molecules. The magnetic force is similar to the force generated in a moving coil in a permanent magnetic field such as in a generator where the mechanical energy of the coil is turned in electrical energy. In this form of magnetophoresis the chemical energy of diffusion is turned into more rapid movement of the molecules.

By this invention, a systematic method has been developed to use these techniques to control the movement of drug molecules into the sub-dermal tissue. Pain relieving drugs were studied using this method because of their applicability to a wide spread number of disease and pain relief from surgical procedures.

The methods as described herein teach the matching of these fields to the structure of the drug. Also, the devices themselves that are used to enhance the movement are different than normal static electric current (iontophoresis) or single magnet magnetophoresis devices that have been used by others, as explained below.

The magnetic devices used for transdermal application of drugs can relieve pain when used by themselves. The use of magnetic devices for pain relief is described, for example, in U.S. application Ser. No. 11/469,346 [US 2008/0103350 A1], U.S. Pat. No. 6,776,753, U.S. Pat. No. 5,312,321, and U.S. Pat. No. 6,461,288, owned by the assignee of the present invention. Thus, the magnetic field strength (milliTesla (mT) or Gauss) is sufficient both to relieve pain as well as to enhance the transdermal movement of the drug. The devices also have a lateral gradient of the field with distance (mT/mm) sufficient to accelerate the movement around obstructing molecular structures.

Conventionally iontophoresis uses static electric fields and the method taught in this invention uses the fields of Transcutaneous Electric Nerve Stimulators (TENS) devices which are widely used for relief of pain. These devices use pulsed electric fields rather than static fields and the systems have been shown to be very effective in the present method in conjunction with the static magnetic field.

The method for drug delivery relies on the determination of the relative movement characteristics of molecules in the subject fields. These are related to the field strength and vector direction of the electric and magnetic fields separately to target the desired area of application.

Diffusion

When a transdermal drug is placed on the surface of skin, the drug, naturally, begins to move into the skin, predominantly along lipid channels between cells. The motion occurs because of the chemical driving force due to the increased concentration on the surface of the skin. As the drug is depleted on the surface and becomes spread out along the direction of motion, the driving force is reduced and the drug “slows down”. This phenomenon of diffusion or dispersion is a natural effect and has been studied in many fields for many decades.

The simplest case to consider is when the drug experiences minimal absorption by the cells as it penetrates the dermal layer. For this case a useful mathematical solution to the diffusion problem is relatively simple. This case is used to illustrate the principle of how the electric field or magnetic field can accelerate the drug.

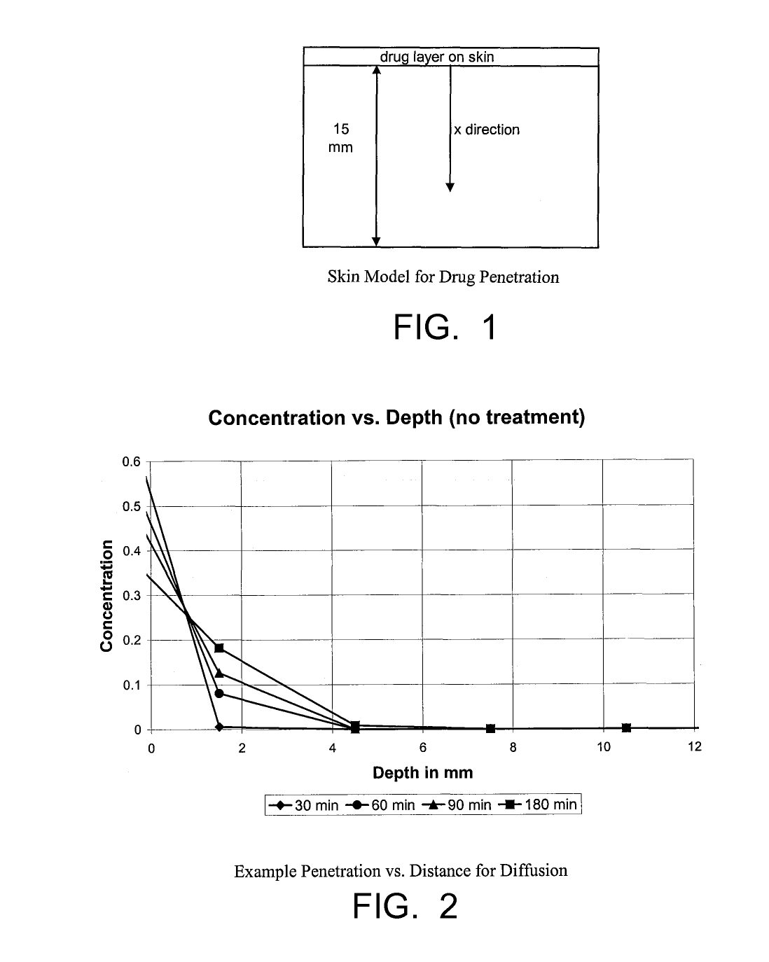

FIG. 1 depicts a simple model for placement of a drug on the surface of the skin. The direction down into the skin in shown as the x axis. If the drug covers most of the skin above the downward arrows then the lateral dispersion to either side of the x axis (e.g. the y axis) is not very important.

The movement of the drug in the x direction can be expressed by the following model solution to the diffusion equation. This is described in many references and the simplest reference that eliminates some of the mathematics on how it is derived is given in “Worked Problems in Applied Mathematics” by N. N. Lebedev, I. P. Skalskaya and Y. S. Ufland, Dover Publications, 1979, page 82.

$$c ( x , t ) = {C}_{o} [ \begin{matrix}1 - \frac{c}{a} - \\ \frac{2}{π} ( {\sum }_{n}^{∞} \frac{sin ( \frac{n π c}{a} )}{n} {e}^{\frac{- {n}^{2} {π}^{2} Dt}{{a}^{2}}} cos ( \frac{n π x}{a} ) )\end{matrix} ] \qquad Equation [ 1 ]$$

Equation [1] is presented to help define the variables and to aid in understanding the physics. The initial concentration of the drug on the surface is C₀. The equation estimates the concentration of that drug at a distance x below the surface (See FIG. 1) for any time t after the drug is applied. The parameter “c” is the distance into the skin to the “target” position for application of the drug and “a” is that distance plus the thickness of the drug layer on the surface. Thus, a-c is the drug layer thickness. In this example the drug layer thickness is 1 mm and the first 15 mm or so of dermal thickness is examined.

As the drug penetrates into the skin the concentration of the drug is depleted from the surface. The drug becomes spread out over the profile distance x. Controlling the time it takes and the concentration gradient is an important part of transdermal application. The key feature of Equation [1] and in controlling how the drug is delivered is the parameter D. This is the dispersion coefficient also called the diffusion coefficient in some cases. The dispersion coefficient in this example is only along the x direction and is sometimes referred to as the longitudinal dispersion coefficient. In the more general case the situation is more complicated as the drug can also disperse laterally and we have a lateral or transverse dispersion. Subsequently one can refer to the dispersion in the longitudinal direction as Dx and in the transverse direction as Dy.

A closer look at the exponent of Euler's number (e) in Equation [1] will aid in understanding the units involved.

−n²π²Dₓt/a²

In the above expression t has the dimensions of time (e.g. minutes) and “a” has the dimensions of length (e.g. mm). In order for this term to be dimensionless in the exponent Dx has the typical “diffusion coefficient” units of area/time (e.g. mm²/minutes).

The function of the electric field and/or the magnetic field is to modify Dx and Dy. The modification of Dx increases the depth of penetration in the required time. The modification of Dy, primarily with the magnetic field, increases Dy also. One of the surprising and novel results is that the magnetic field increases Dx (in the direction of the diffusion of the molecule) more than Dy (perpendicular to the magnetic field, the direction of the magnetic force on the molecule). This novel and surprising result supports and explains one of the embodiments of the invention.

FIG. 2 is a series of curves developed at various times for plain diffusion for this example. This is typical of data that is obtained if the drug or chemical of interest is analyzed at different depths at different times. The section to the left of zero on the x axis is the concentration of chemical in the applied layer.

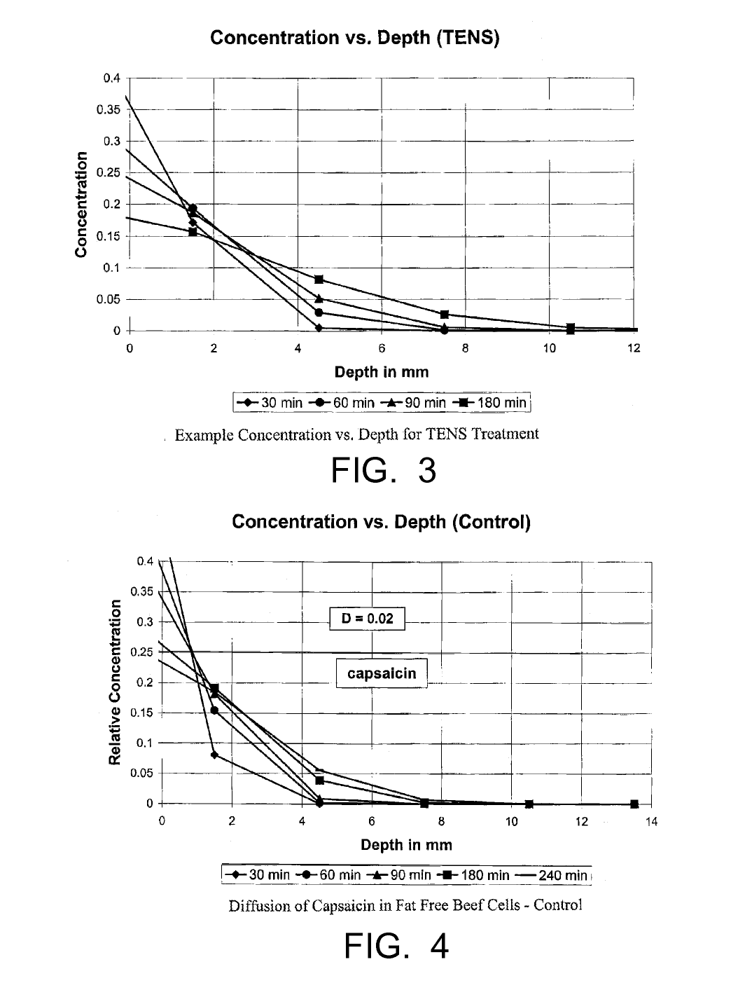

FIG. 3 is an example of the results from the same drug or chemical using a TENS unit to accelerate the movement of the chemical based on measured concentration and distance. All of the parameters in Equation [1] have been taken the same, but Dx has been increased by 5 times in the presence of a TENS unit. At the longest time (180 minutes) the concentration at 10 mm is the same as at 5 mm in FIG. 1. The effect of using a magnet on Dx alone is somewhere between these two extremes but closer to FIG. 3 than to FIG. 2.

This type of analysis of the concentration as a function of distance from chemical analysis of experimentally applied drugs can pinpoint the relative rates of movement of various drugs. The application of a known amount of a drug under known magnitudes of magnetic and electric fields can be analyzed by measuring the concentration of the drugs at various positions in the skin at various times after application. This data then allows one to construct the most beneficial paradigm for the applications of the drugs.

The actual measurement of the concentration profiles are more difficult when carried out in humans, but similar results occur when studies are performed on a wide variety of tissue. G. B. Kasting, W. R. Francis, L. A. Bowman and G. O. Kinnett (“Percutaneous absorption of vanilloids: in vivo and in vitro studies”, Journal of Pharmaceutical Sciences, 1997 Volume 86(1) pages 142-6) measured the rates of capsaicin analogs in live rats, excised skin from dead rats and human cadaver skin. While rat skin was more permeable than human skin by a different amount for each capsaicin analog, the measured rank order stayed the same in all cases including the human skin. I. Boudry, O. Blanck, et al. (“Percutaneous penetration and absorption of parathion using human and pig skin models in vitro and human skin grafted onto nude mouse skin model in vivo”, Journal of Applied Toxicology, 2008 Volume 28(5) pages 645-57) found that the in vitro models closely predicted the in vivo human volunteer absorption. The net result of this is that it becomes possible to make measurements in any reasonable skin model system and then to correlate those measurement with human measurements to “calibrate” all the factors.

Based on the above, it can be seen that measurements made on model tissue will predict the benefits of either the magnetic field alone, or in combination with the electric field. The final application in humans in terms of the relative order of movement can thus be assured before exact values are obtained. The exact values can be obtained from blood samples to calibrate the test systems.

Magnetic and Electric Properties

Molecules are made up of atoms wherein the nucleus is positively charged and the atoms are surrounded by swarms of the negatively charged electrons. Complicated molecules, like pharmaceuticals or drugs, are systems of atoms held together by very complicated interactions of the electrons. There are only a few atoms, like helium and neon (so-called “inert gases”) where the distribution of electrons is such that they do not form many “bonds” with other atoms. When atoms are put together into molecules, frequently the charges will not be uniform in space and the spatial arrangement results in a partial charge separation leading to a property known as the “dipole moment”.

The electric field moves particles in a solvent if those particles have a charge, either full charges like “ions” or partial charges like those molecules with a dipole moment. The speed with which the molecules will move is directly proportional to the square of the dipole moment. The speed is also directly proportional to the magnitude of the electric field and inversely proportional to the viscosity of the solvent and the square of the size of the molecule.

The electric field also induces changes in the electron distribution based on a property known as the “polarizability”. This is related to what is called an “induced dipole moment”. The movement of the molecule is also directly proportional to this property because it represents a further partial charge on the molecule. The movement of the molecule is proportional to the polarizability and to the square of the dipole moment. This means that the dipole moment has four times more influence than the polarizability for moving the molecule if the two properties are of about equal magnitude. In the present invention, the “solvent” is the lipid layer between the cells in the tissue of the mammal.

The important features for the movement by an electric field are related to the molecule itself, the velocity of movement is directly proportional to the polarizability and the square of the dipole moment. The velocity is inversely proportional to the square of the molecule size. The rest of the properties that control how fast the molecule moves are related to the solvent. For transdermal applications the matrix of lipids and cellular structures that make up tissue may be considered the solvent.

There are analogous properties for the magnetic field. There is a “magnetic moment” that corresponds to the dipole moment and a magnetizability corresponding to the polarizability. A difference is that not many molecules have a permanent magnetic moment, including all of the drugs presented in this invention. The permanent magnetic moment is related to structures with unpaired electrons such as occur in some metals, metallorganic complexes and certain organic matter such as “radicals”. Thus, for moving the drugs of interest one is basically relying on the induced magnetic moment of magnetizability. This means that the magnetic effect is about 4 times weaker than the effect due to the electric field. Surprisingly, in the present invention, this magnetic effect works better than one would expect and the effects of the field are less harmful than energy derived from an electric field.

The magnetic effect has another difference. The magnetic field moves the molecules (or ions) perpendicular (or sideways) to the field. It causes the complex drug molecules to twist and move sideways as well as to move forward. The electric field causes the molecules to align with the electric field and penetrate faster while the magnetic field causes them to disperse laterally. The twisting action can however cause the molecules to move forward and the relative contribution of the movement is unknown and cannot be predicted. Unexpectedly we have found that while we cannot predict the specific lateral motion, the method as described herein allows one to predict the effect the lateral motion has on the movement into the skin. The importance and the advantage of the magnetic field is that the molecules can more easily move around obstacles such as cell walls of the tissue and into the depth of the sub-dermal area.

The magnetic field also has another attribute. The energy for the movement of molecules in an electric field comes from the energy in the field itself. The energy is derived from the power source. In the magnetic field the energy is derived from the concentration gradient that is already moving the molecule. The magnetic field is “permanent” (actually extremely long lived with slight losses over long periods of time due to entropic effects) and the energy does not come from the magnet. To use an electric generator as an example a coil of wire is moved through permanent magnets to create a flow of electrons in the wire. The magnets cause the electrons to move but the energy that actually is translated to the movement of the electrons comes from the movement of the wire coil. This is how mechanical energy from a turbine, for example, is converted into electrical energy. The energy does not come from the magnetic field.

Thus, the use of magnets in magnetophoresis drug delivery has the added advantage that the device is permanent and does not require a source of electrical power. An electromagnet could be used, for example, but one of the benefits for moving drugs with magnets is the use of a simple compact device that can be used repeatedly in many environments by non-medical personnel.

The skin or mucosa functions as the primary barrier to the transdermal penetration of materials into the body and represents the body's major resistance to the transdermal delivery of therapeutic agents such as drugs. To date, efforts have been focused on reducing the physical resistance or enhancing the permeability of the skin for the delivery of drugs by passive diffusion. Various methods for increasing the rate of transdermal drug flux have been attempted, most notably using chemical flux enhancers. Some approaches to increase the rates of transdermal drug delivery include use of alternative energy sources such as electrical energy and ultrasonic energy. Electrically assisted transdermal delivery has also been referred to as electrotransport or iontophoresis. The drug delivery is induced or aided by application of an electrical potential. These electrical devices provide a static electrical field and the TENS unit described herein provide a pulsed electrical field; i.e., the electrical field is not on constantly. However TENS is used to relieve pain and to cause muscle stimulation and thus the use of TENS with the magnets as described herein can provide a dual benefit of pain relief while there is dermal or mucosal transport of drugs.

Specific Chemicals

One of the uses for both magnets and TENS units is for the treatment of pain. Chemicals used in the treatment of pain are used as the exemplary models for the method taught herein. The following examples used capsaicin, lidocaine, bupivacaine, ketamine and ketorolac. Since chemicals can undergo metabolism and degradation during transdermal infusion another important part of the process is to understand the chemistry of the metabolism of degradation. The infusion time should be fast enough so that the drugs are delivered relatively intact to the pain site at the desired distance below the surface of the skin. For other types of drugs such as vaccines and antibiotics one would want to transfer them relatively unchanged in the blood stream for distribution throughout the body.

The Method and The Apparatus Used

The method as taught controls the transdermal rate of movement of chemicals by controlling the magnitude and direction of the magnetic and/or electric fields respectively.

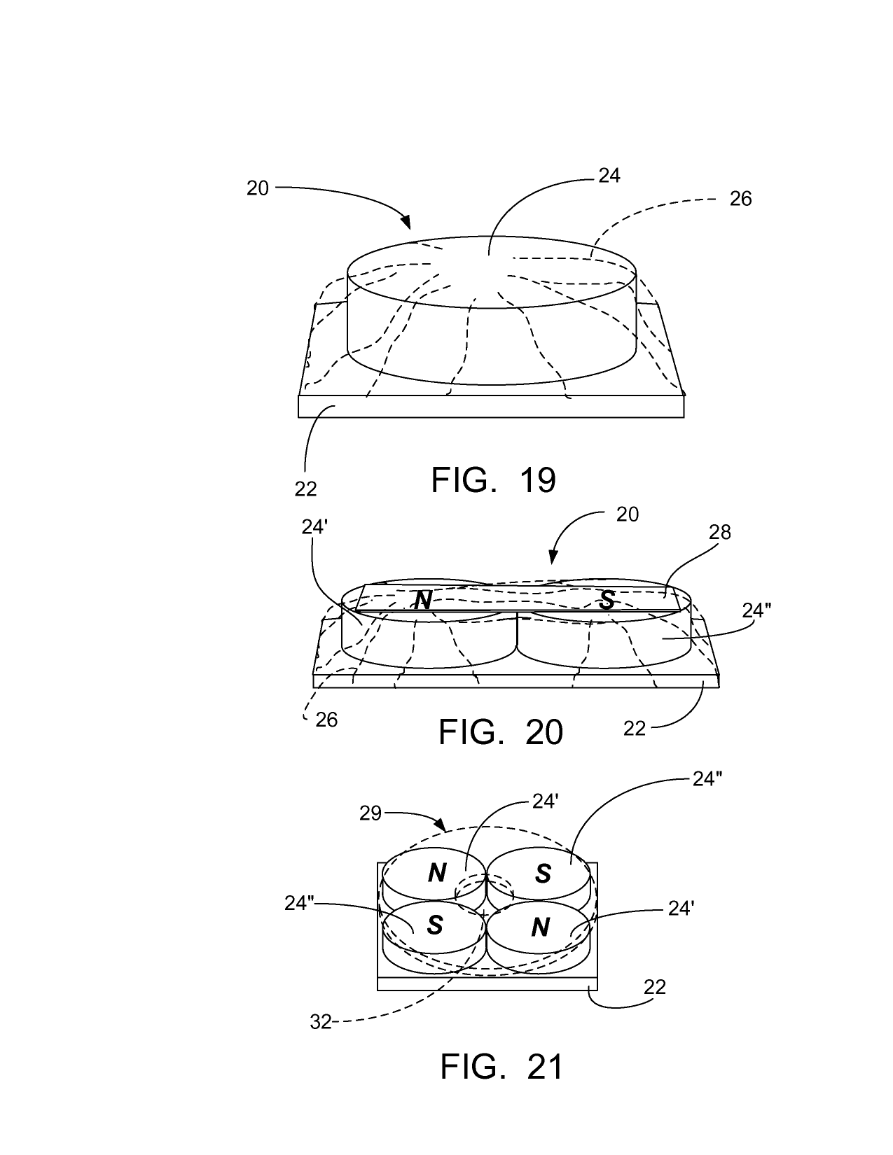

The direction of the magnetic field augmented movement is controlled by placing the magnet (or magnets when multipolar magnets such as quadrapolar magnet array are used) above the chemical with the plane of the surface of the magnets perpendicular to the desired direction of movement. This type of apparatus is seen in FIGS. 19 to 22 and is described below. FIG. 23 provides an illustration of the action of the magnetic field on the drug molecules.

The direction of the electric field augmented movement is controlled by aligning the positive and negative electrodes of the TENS unit parallel to the desired direction of movement above and below the placement of the chemical on the skin.

When both the magnet array and the TENS unit are used the magnet(s) are placed over the electrode that is placed over the chemical or drug.

A sufficient amount of the drug is placed below the electrodes or magnet array such that when it is distributed into the body it has the effective amounts as prescribed for that drug.

The magnitude of the magnetic field is matched to the magnetic dipole moment (if any) and the magnetization of the chemical to increase the movement by the desired amount. The desired amount is that amount which distributes the drug into the skin in the desired time at the desired concentration. All of these values are easily determined experimentally for each drug using the same analytical techniques as for the electric field or for diffusion.

The magnitude of the electric field is matched to the dipole moment and polarizability of the drug to increase the rate of movement by the desired amount. The desired amount is that amount which distributes the drug into the skin in the desired time at the desired concentration. All of these values are easily determined experimentally for each drug in laboratory experiments which then can be calibrated for use in mammals by correlation with known transdermal delivery rates or by blood level measurements in a few initial cases.

The drugs are formulated in a medium that allows for safe application to the skin without rash or reaction. The medium can also be isotonic to the skin so as not to cause water loss or swelling. The medium can be compatible with tissue. Glycerol is a typical solvent for such a system but the medium can also contain preservatives, stabilizers or other adjuncts, and other solvents, creams or carriers can be used. These chemical solvents are well known to those skilled in the art and multiple examples may be found in sources such as “Dermatologic, Cosmecetic, and Cosmetic Development edited by Kenneth A. Walters and Michael S. Roberts Table 1, page 502 (CRC Press 2007).

Referring now to FIG. 19, the diagram illustrates the features of an exemplary implementation of static magnet device 20 using a single magnet 24, positioned on a pad substrate 22, containing a drug formulation. The magnet 24 is held in place by such as a cover with a restraining pouch 26. The pad substrate 22 contains the drug formulated in a medium which allows dispersion transdermally. The pad substrate 22 side that doesn't contain the magnet 24 is placed on the dermal surface D of the mammal to be treated. This is illustrated in FIGS. 19 through 22. An exemplary medium would be isotonic formulation. The treatment apparatus including pad may be held on by adhesive tape, or other attachment mechanism.

In another exemplary implementation illustrated in FIG. 20 two magnets 24′, 24″ are utilized. One magnet 24′ is North facing and the second magnet 24″ is South facing. Optionally a metal conductive plate 28 may be placed across the tops of the magnets 24′, 24″ and the restraining pouch cover 26 placed over the magnets and plate 28 to increase the skin directed field.

In an additional exemplary implementation in FIG. 21, the diagram illustrates the use of a quadrapolar magnet array 29 made up of magnets 24″, 24″ with the pad substrate 22. As in the earlier exemplary illustrations, the pad substrate contains a drug formulation for use in transdermal medication. The diagram in FIG. 21 illustrates the use of a flux ring 32 (in dotted form) held in place with the restraining pouch cover 26.

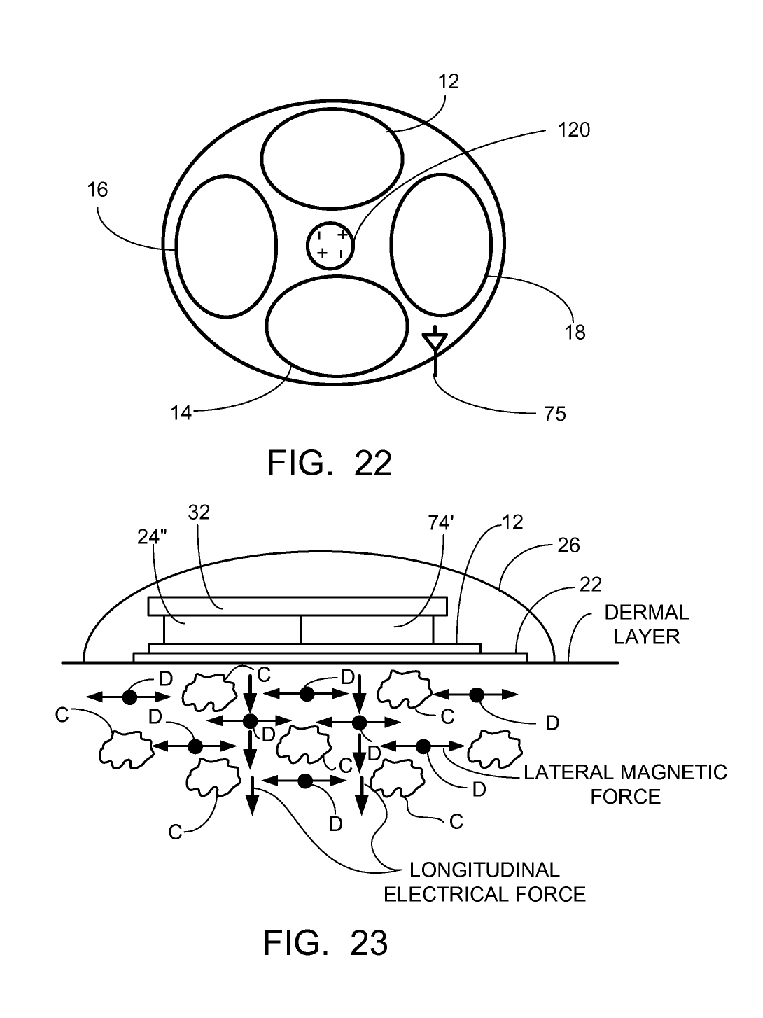

FIG. 22 illustrates an alternative arrangement of a TENS/QMA array wherein the TENS electrodes 12, 14, 16 and 18 are arranged to surround the QMA 120 on the drug pad 22. U.S. patent application Ser. No. 11/678,528 [2008/0207986 A1], commonly owned with the present application, illustrates and describes the use of such embodiment, including a field monitoring antenna 75 and related apparatus to regulate the electrical field produced by the TENS apparatus.

Those skilled in the art are directed to previously identified use of magnetic devices for pain relief-described, for example, in U.S. patent application Ser. No. 11/469,346 [2008/-103350 A1], U.S. Pat. No. 6,776,753, U.S. Pat. No. 5,312,321, and U.S. Pat. No. 6,461,288 for examples of the construction and use of QMA's in the treatment of pain. It has been determined that the magnetic field strength (milliTesla (mT) or Gauss) is sufficient both to relieve pain and to enhance the transdermal movement of the drug. The devices incorporating the TENS application also have a lateral gradient of the field with distance (mT/mm) sufficient to accelerate the movement around obstructing molecular structures.

The figures as described above are illustrations of potential arrangements of pad substrates and magnets and accordingly it is not intended that the scope of the disclosure in any way be limited to the above illustrations.

The use of a TENS device is described in U.S. application Ser. No. 11/678,528 [2008/0207986 A1] which is incorporated by reference herein. The movement of the drug by the magnet(s) experiences an additive effect from the electric field which is controlled by aligning the positive and negative electrodes of the TENS unit parallel to the desired direction of movement above and below the placement of the chemical on the skin.

When both the magnet array and the TENS unit are used the magnet(s) are placed over the electrode that is placed over the chemical or drug.

A sufficient amount of the drug is placed below the electrodes or magnet array such that when it is distributed into the body it has the effective amounts as prescribed for that drug.

### EXAMPLE A

### Magnet Array

The parameters for the quadrapolar magnetic array used in the obtaining the experimental data were:

Each individual magnet had:
a residual induction value of at least 1,500 mT (magnetization of 1,200,000 amp/meter);
diameter of about 12.7 mm; and a
thickness of about 6.35 mm.

The magnets were arranged in an alternating polarity sequence on an iron flux ring (the iron flux ring is shaped like a washer). This positioning increased the field in the direction away from the flux ring. This arrangement is similar to the standard QuadraBloc® sold by Gradient Technologies, LLC. The magnetic field at the center of a North Pole of the magnets was about 530 mT. The array was placed over the drug such that the face of the magnets (away from the flux ring and towards the skin) covered the drug solution applied to the test material or skin. The Quadrapolar magnet array is given in FIG. 18 and the Quadrapolar magnet array with a flux ring is shown in FIG. 19. This configuration is described in U.S. applications Ser. No. 11/469,346 which is incorporated by reference herein.

### EXAMPLE B

### TENS Unit

The TENS unit used in the following experiments was set for 20 volts with a pulse rate of 100 Hz and a pulse width of 200 microseconds. Other usable settings will influence the results as the strength of the field is directly proportional to all of the variables and increasing any of the variables will drive the drug faster. The settings are important for treating underlying pain; however, when drugs are being applied at the same time as the TENS is being utilized to treat pain, it generally is not desirable to use the highest settings. The highest setting can cause excessive numbness and muscle twitching. For drug delivery along with pain relief one would use parameters that provided the treatment without overstimulation of nerves and muscles.

The settings utilized in the described experiments were chosen as initial points to determine relative diffusion rates. Once the relative rates are determined in the test system both the electric or magnetic field strength can be adjusted as required for each formulation or drug.

### EXAMPLE C

### Tissue Test Samples

The test system used to determine the rates of dispersion in tissue was commercial homogenized beef tissue with various levels of lipids (fat) to cells compressed into 3 mm sheets. Human skin tissue contains from 1-7% lipids (Marilyn Lampe et al., J. Lipid Res. 1933, 24: 120-130). The range of lipids in the beef tissue used in the examples thus span the range found in human tissue. The beef tissue was purchased with selected levels of fat present in the samples as measured by the producer. 21 CFR 101.62(b) lists the requirements for producers to be able to label commercial homogenized beef tissue as low fat or fat free. There were differences due to temperature, age of the homogenized beef tissue, and the contact between the beef and the pads but in no case was there a difference in the rank order of the results or the diffusion coefficient for a specific drug. This type of homogenized beef tissue is sold as bologna of various types and various makes. A stack of five sheets (5 slices) had a height of 15 mm and the stack of sheets was arranged into various areas. The drug or formulation to be tested was placed on top of the tissue stack and then after a specified time the stack was separated and the individual 3 mm layers (bologna sheet/slice) were analyzed for the drug content. The drug migration was analyzed by extracting the tissue at the various depths with solvents and then quantifying the drug in that sample. A general method is the extraction of the tissue with reagent grade methanol (Aldrich Sigma Chemical Company) and then the use of liquid chromatography (LC) calibrated for the particular drug. The analysis were run on one or the other of two different Shimadzu LC with UV detectors and there were no instrumental effects on the data. For a typical drug at a given concentration, there were three stack arrangements made. The stacks were used for (1) control sample (simple diffusion sample with no external driving force), (2) quadrapolar magnet sample, and (3) TENS unit sample.

### EXAMPLE D

### Drug Formulation

The drugs used in the following experiments were formulated in a medium that allowed for safe application to the skin without potential rash or reaction. The medium can be isotonic for the skin to prevent either water loss or swelling. The medium can also be compatible with the tissue. Glycerol is an example of a typical solvent for such a system but the medium can also contain preservatives, stabilizers or other adjuncts and other solvents, creams or carriers can be used and are known to those of ordinary skill in the art.

### Example 1

### Capsaicin

### 1.A. Fat Free Tissue

A solution of 0.075% capsaicin in a glycerol base was applied to fat free beef cell tissue prepared as in Example C. The capsaicin formulation was applied to the top surface of three (3) stacks, 15 mm in height. Each stack consisted of five 3 mm segments. There were three tests: one per stack of fat free beef cell tissue. The tests were 1) Control with no applied field; 2) a magnetic field applied; and 3) TENS field was applied as given in Example C with the parameters for the magnets and the TENS unit as given in Examples A & B. The test for the diffusion sample and the magnetic field sample ran for 4 hours and the TENS unit sample ran for 1 hour. Each stack was sacrificed when tested at the selected interval (30 mins., 60 mins, 90 mins., 180 mins. and 240 mins.) to ascertain the rate of movement of the test material through the beef tissue. Thus there were multiple stacks used for each of the three tests. All three tests were replicated.

At the end of the test period each 3 mm section was extracted with methanol and the concentration of capsaicin measured by liquid chromatography. The ratio of the capsaicin in each layer compared to the initial concentration (0.1 grams of the 0.075% solution) was used in conjunction with Equation [1] to calculate the diffusion coefficient Dx.

The relative concentration versus the depth profile is given in FIG. 4. The experimental diffusion profiles were consistent with the diffusion coefficient of 0.02 mm²/min for this tissue type.

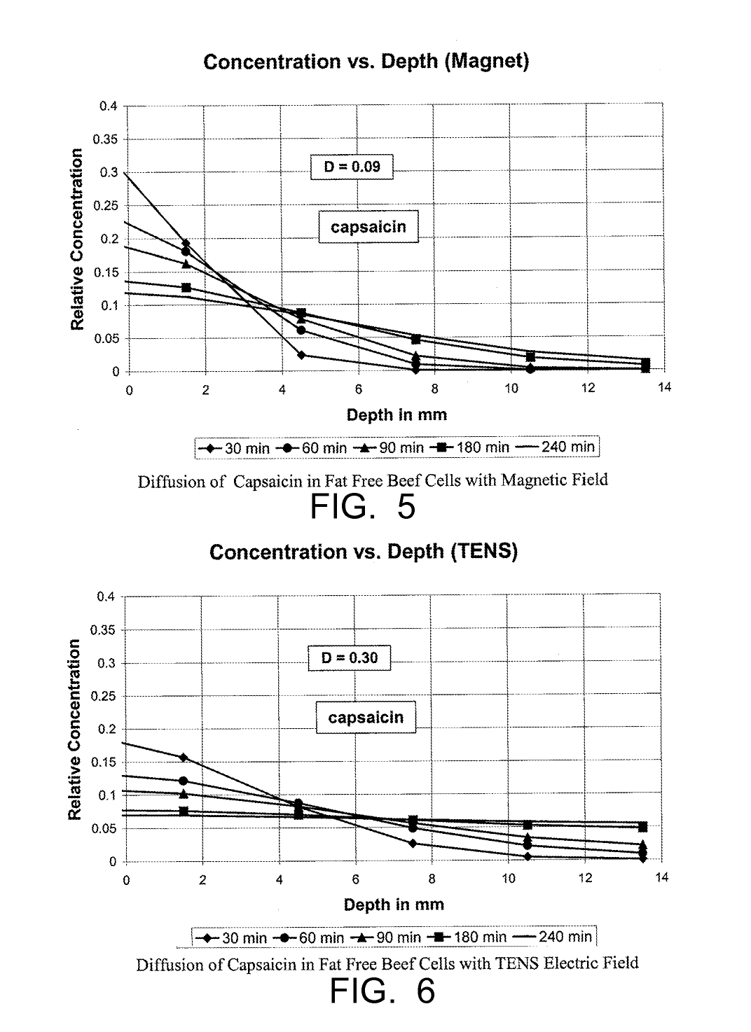

FIG. 5 shows the data where the movement of the drug is augmented by the applied magnetic field. The measured field strength varied from 530 mT at the surface (top of the stack) to 46 mT in the bottom section. Example E describes the technique used to determine the field strength. The Dx for the magnetic field augmentation is 0.09 mm²/min. The diffusion coefficient was enhanced 4.5 times. The diffusion coefficient is the definitive parameter for comparison. The data can also be described in simpler terms. FIG. 4 shows the drug moved 4.5 mm after 30 minutes through simple diffusion and required 240 minutes to diffuse to a depth of 8 mm. In the second test sample (FIG. 5) the drug penetrated to a depth of 7.5 mm in 30 minutes and by the end of 240 minutes had penetrated the total tissue system (15 mm) with the addition of the magnetic field to the second test sample. The magnetic field doubled the rate of penetration of the drug in the fat free sample.

FIG. 6 presents data for the augmented movement due to the TENS electric field. Whereas the magnetic field uses energy from the normal diffusion motion the electric field actually imparts energy from the field and the diffusion coefficient Dx was 0.30 mm²/minute. This is 15 times that of the control. In terms of absolute penetration the front had moved 12 mm in the first 30 minutes and totally penetrated the height of the stacks in 60 minutes after which it begins to level out across the stacks. The final state of a diffusion system like these is the uniform distribution across the tissue. There would be differences in actual mammalian systems as once the drug enters blood vessels the drug would be carried away from the penetration site. The relative diffusion rates are more accurately measured in this type of system because the results are not confounded by the blood transport effects while such effects can be taken into account in a transport model that is slightly more complicated that the one used here (Equation [1]).

The purpose is to deliver the drugs to either the tissue or to blood (depending on the drug). For a local anesthetic, however, it may not be desirable to move the drug into the blood stream as it could potentially be cleared by the body too fast. Thus, for anesthesia magnetic enhancement could be more desirable because the rate is faster than diffusion alone but slow enough to control and the device requires no external source of power.

### 1.B. 15% Fat Tissue

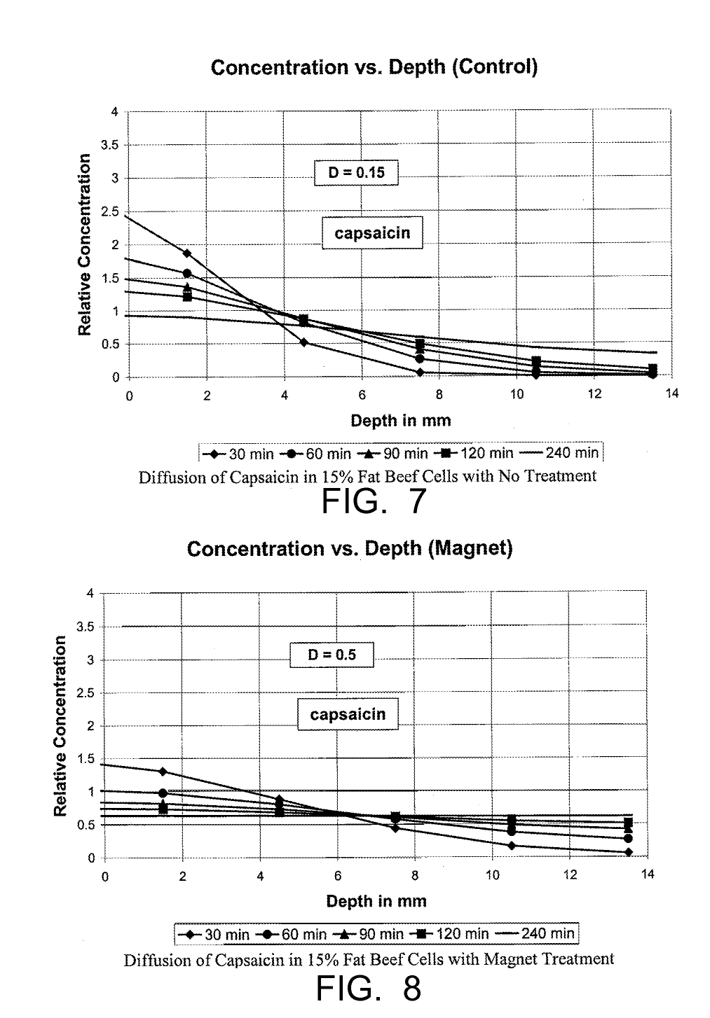

One gram of 0.075% solution of capsaicin in a glycerol base was applied to beef cell tissue with a fat content of 15% prepared as in Example C. The capsaicin formulation was applied to the top surface of three (3) stacks, each 15 mm in height. Each stack consisted of five 3 mm segments of beef cell tissue. There were three tests: one per stack of 15% containing fat beef cell tissue. The tests were 1) Control with no applied field (FIG. 7); 2) a magnetic field applied (FIG. 8); and 3) TENS field (FIG. 9) applied as given in Example C with the parameters for the magnets and the TENS unit as given in Examples A & B. The test for the diffusion sample and the magnetic field sample ran for 120 minutes and the TENS unit sample ran for 1 hour. The diffusion times were shorten as there was more rapid movement of capsaicin in the higher lipid content tissue. As in Example 1.A. there were multiple stacks of the 15% containing fat beef cell tissue used for each of the three tests: control, magnetic field and TENS unit. All three tests were replicated.

At the end of the test period each 3 mm section was extracted with methanol and the concentration of capsaicin measured by liquid chromatography. The ratio of the capsaicin in each layer compared to the initial concentration (1 grams of the 0.075% solution) was used in conjunction with Equation [1] to calculate the diffusion coefficient Dx. In the figures for the fat containing tissue the ratio of concentrations has been multiplied by 10 to distinguish this data from the data in Example 1.A (fat free tissue) since 10 times more material was applied. This is only to aid in plotting as the amount used does not change the ratios of concentrations at the different levels unless the diffusion coefficient is a function of the amount applied. This event is rare. FIG. 7 exhibits the control sample diffusion of capsaicin in the 15% fat containing beef tissue. The diffusion coefficient (0. 15) is about 8 times higher in the lipid containing tissue than it was in the lean tissue (0.02, FIG. 4). FIG. 8 exhibits the effect of applying a magnetic field under the same conditions. The diffusion coefficient of 0.50 with the application of magnets is 3 times greater than simple diffusion of the material alone. Application of the TENS appeared to have a minimal effect on the diffusion coefficient (0.55, FIG. 9).

Human skin tissue contains from 1-7% lipids (Marilyn Lampe et al., J. Lipid Res. 1933, 24: 120-130). The range of lipids in the beef tissue used in the examples thus spanned the range found in human tissue.

### Example 2

### Lidocaine

A hundred microliters of a 4% lidocaine was applied to low fat beef cell tissue prepared as in Example C. The lidocaine formulation was applied to the top surface of three (3) stacks, 15 mm in height. Each stack consisted of five 3 mm segments. There were three tests: one per stack of fat free beef cell tissue. The tests were 1) Control with no applied field; 2) a magnetic field applied; and 3) TENS field was applied as given in Example C with the parameters for the magnets and the TENS unit as given in Examples A & B. The tests for the diffusion sample (control), the magnetic field sample, and the TENS unit sample ran for 6 hours. As in Example 1 for capsaicin the testing was destructive and multiple stacks were used for each test of 1) control, 2) magnetic field applied, and 3) application of TENS field. All three tests were replicated.

At the end of the test period each 3 mm section was extracted with methanol and the concentration of lidocaine was measured by liquid chromatography. The ratio of the lidocaine in each layer compared to the initial concentration (100 microliters of a 4% solution) was used in conjunction with Equation [1] to calculate the diffusion coefficient Dx.

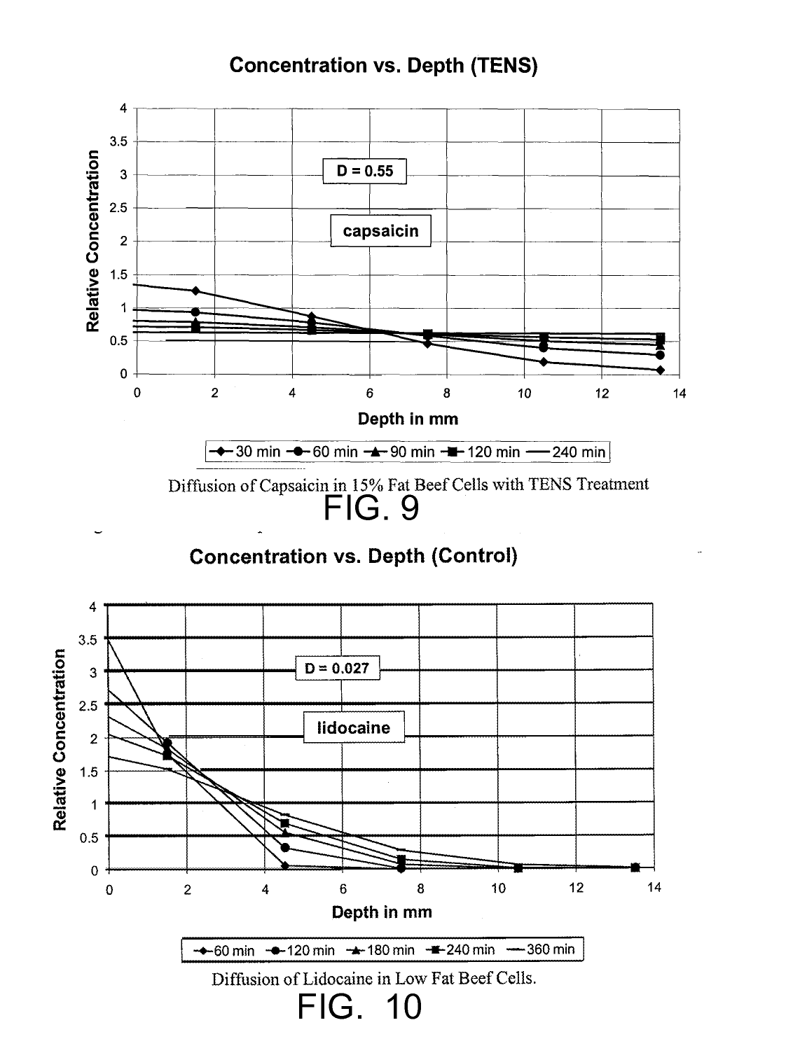

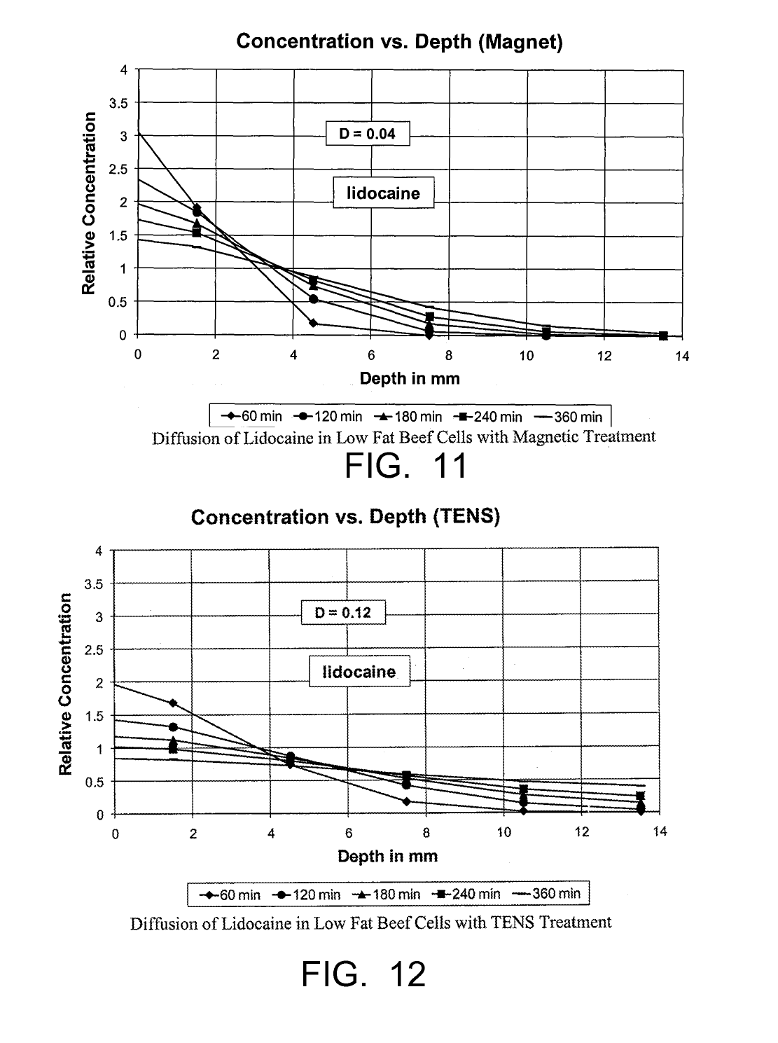

In FIG. 10 it is shown that lidocaine exhibited a diffusion coefficient of 0.027 in the control test. When magnets were applied to the test sample there was an increase of 48%, or about 1.5 times, in the diffusion coefficient to 0.04 mm²/min (FIG. 11). The TENS treatment alone gave a diffusion coefficient of 0.12 mm²/min (FIG. 12) for an increase of 4.4 times from the control sample's diffusion coefficient. These results demonstrate the value of the method of determining and selecting field strengths.

### Example 3

### Bupivacaine, Ketorolac and Ketamine

Similar experiments as in Example 1 & 2 were performed with bupivacaine, ketorolac and ketamine, both singly and in combination. Qualitatively there were increased values in the range of 4-5 times for the magnetic treatment test, and 6-8 times for the TENS treatment test versus the control test. The biochemical substances in the beef cells in the LC measurement range of these 3 drugs caused considerable scatter in the data. To obtain precise figures similar to those shown for capsaicin and lidocaine requires a different analytical procedure wherein potentially a different extractant is used with potentially a modified method.

### Example 4

### Insulin

Similar experiments as in Example 1 & 2 were performed with insulin. There are many forms of the polypeptide generally called insulin that are used in the treatment of diabetes. The protein portion of the active section of the molecule has a molecular weight of approximately 6,000 (Daltons). The natural products contain small amount of zinc, nickel, cobalt, or cadmium. Some formulations including the one used in this experiment contain added zinc as the chloride.

A hundred microliters containing 100 IU of insulin as insulin glargine (Lantus®) was applied to the top surface of three (3) stacks, 15 mm in height. Each stack consisted of five 3 mm segments. There were three tests: one per stack of fat free beef cell tissue. The tests were 1) Control with no applied field; 2) a magnetic field applied; and 3) TENS field was applied as given in Example C with the parameters for the magnets and the TENS unit as given in Examples A & B. The tests for the diffusion sample (control), the magnetic field sample, and the TENS unit sample ran for 12 hours. As in Example 1 & 2 the testing was destructive and multiple stacks were used for each test of 1) control, 2) magnetic field applied, and 3) application of TENS field. All three tests were replicated.

At the end of the test period each 3 mm section was extracted with methanol and the concentration of insulin was measured by liquid chromatography. The ratio of the insulin in each layer compared to the initial concentration (100 microliters of a 100 IU solution) was used in conjunction with Equation [1] to calculate the diffusion coefficient Dx.

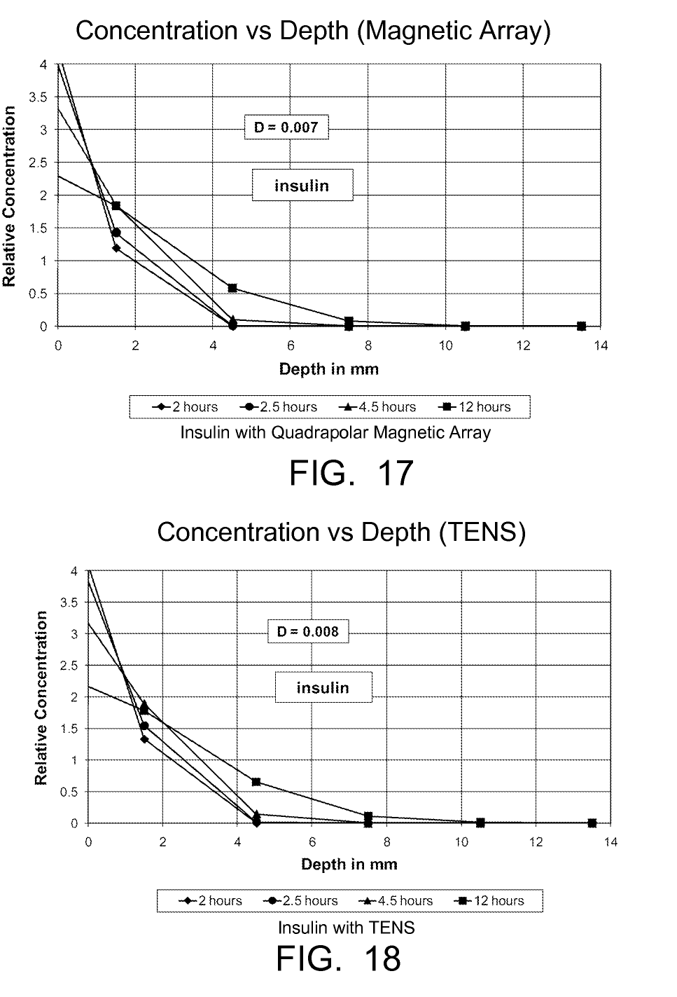

In FIG. 16 it is shown that insulin exhibited a diffusion coefficient of 0.002 in the control test. When magnets were applied to the test sample there was an increase of three and a half times in the diffusion coefficient to 0.007 mm²/min (FIG. 17). The TENS treatment alone gave a diffusion coefficient of 0.008 mm²/min (FIG. 18) for a similar increase from the control sample's diffusion coefficient. These results demonstrate the value of the method of determining and selecting field strengths.

The method as described herein appears to work with any compound for which there is a magnetic moment or an induced magnetic moment.

### EXAMPLE E

### Magnet Field Strength Measurement

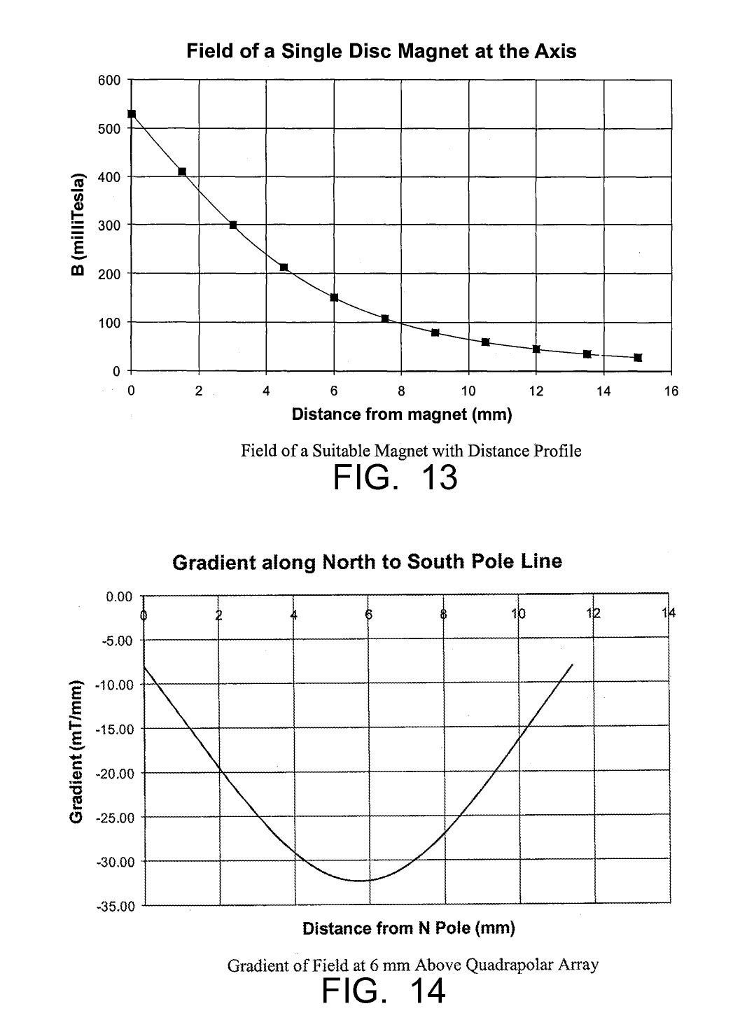

The field strength of the magnets was measured with a field measuring device known as a “Gaussmeter”. The measured field above the axis of a single magnet is shown in FIG. 13. The simulated skin layers in this instant invention are 15 mm thick. The field from a single magnet of the size used herein (see Example A) was 28 mT at 15 mm and 5 mT at 30 mm.

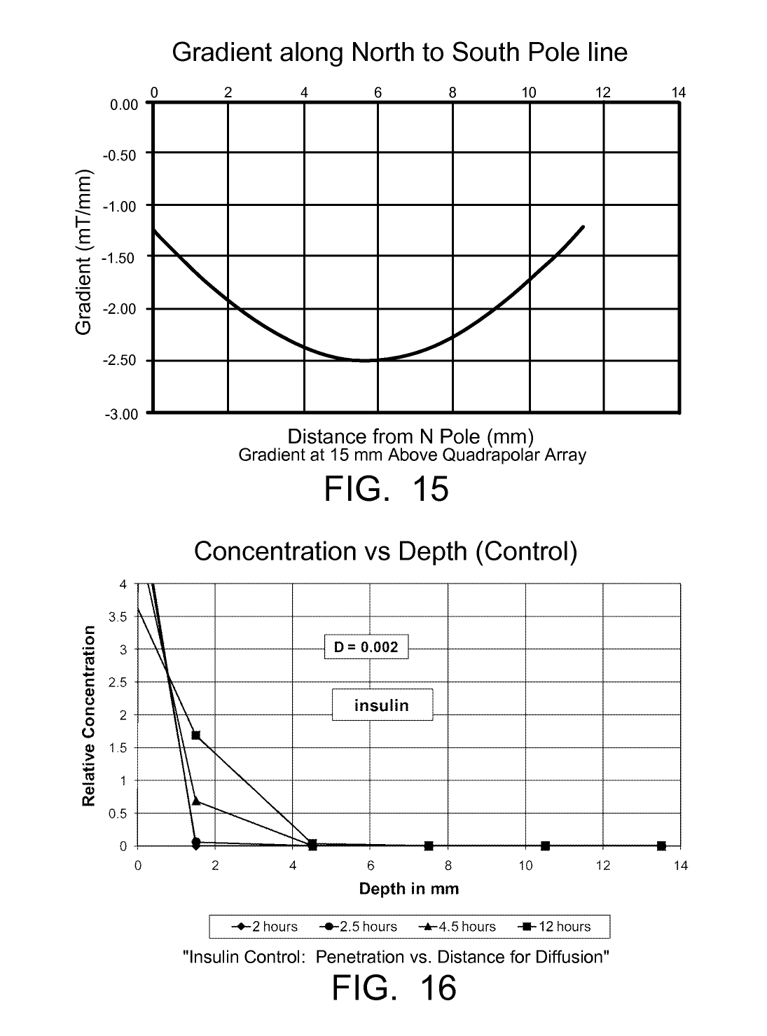

If 2 or more magnets are used in a north—south arrangement, there was an improvement in the measured field versus a single magnet of sufficient field strength. FIG. 14 shows the measured gradient of the field at 6 mm above the magnets. FIG. 15 illustrates the measured gradient at 15 mm above the magnets. The value of the field and the gradient as a function of distance is important to ensure adequate movement of the molecules.

While this disclosure has been described with emphasis on selected implementations, it should be understood that within the scope of the appended claims, the disclosure might be practiced or carried out in various ways other than as specifically described herein. In addition, many modifications may be made to adapt a particular situation, material, composition of matter, method, process step or steps, to the objective, spirit and scope of the present disclosure. All such modifications are intended to be within the scope of the claims appended hereto. In particular, while the methods disclosed herein have been described with reference to particular steps performed in a particular order, it will be understood that these steps may be combined, sub-divided, or re-ordered to form an equivalent method without departing from the teachings of the present disclosure. Accordingly, unless specifically indicated herein, the order and grouping of the steps is not a limitation of the present disclosure. The apparatus and methods disclosed herein include any combination of the different species or embodiments disclosed.

1. A method for administering drugs through a dermal layer comprising the following steps:
providing a magnetic device of at least one magnet to provide an effective magnetic sphere of influence at the desired site of action.
providing a tissue permeable drug formulation which is positioned on the site of action; and
positioning the magnetic device adjacent to said tissue permeable drug formulation
wherein the magnetic sphere of influence of the magnetic field moves the drug though the dermal layer by interaction with the magnetic moment of the drug sufficient to increase the movement of the drug beyond the movement of diffusion alone.

2. The method of claim 1 wherein the magnetic field is at least 1.5 mT at the desired depth of penetration of the drug.

3. The method of claim 1 further compromising the use of iontophoresis as a field enhancer.

4. The method of claim 1 where the drug formulation contains pain-relieving compounds, or active pharmaceutical ingredients, their pharmaceutically acceptable salts or prodrugs thereof.

5. The method of claim 4 wherein the compounds are selected from the group consisting of insulin, capsaicin, ketamine, bupivacaine, lidocaine, prilocaine, zylocaine, articaine, cholorprocaine, mepivacaine, procaine, opiates, ketoprofen, codeine, epinephrine, and ketorolac.

6. The method of claim 1 wherein the drug formulation contains compounds selected from the group consisting of preservatives, stabilizers, creams, gels, and isotonic solvents.

7. A method for administering drugs through a dermal layer, comprising the following steps:
providing a magnetic device in a magnet array of alternating polarity in which the magnetic poles are separated by a predetermined distance to provide an effective magnetic field creating a sphere of influence at the desired site of action from the magnetic fields of all adjacent poles;
providing a tissue permeable drug formulation which is positioned on the site of action; and
positioning the magnetic device adjacent to tissue permeable drug formulation wherein the magnetic sphere of influence of the magnetic field moves the drug through the dermal layer.

8. The method of claim 7 further comprising providing a magnet array of two (2) magnets.

9. The method of claim 7 further comprising providing a magnet array of four (4) magnets.

10. The method of claim 9 wherein four magnets are arranged in alternating polarity on an iron flux ring.

11. The method of claim 7 wherein each of the magnets has a residual induction value of at least about 1,400 mT.

12. The method of claim 7 wherein each of the magnets has a diameter of about 12.7 mm and a thickness of about 6.35 mm.

13. The method of claim 7 wherein the magnetic field at the center of the North Pole of the magnets is about 530 mT.

14. The method of claim 7 further comprising a magnetic field gradient at the site of action of adjacent poles of at least 1.5 mT/mm in the direction perpendicular to the axis from the magnetic poles.

15. The method of claim 14 wherein the magnetic field gradient at the site of action of adjacent poles is at least about 1.5 mT/mm at a distance of 15 mm from the magnetic array.

16. The method of claim 7 further compromising the use of iontophoresis simultaneously or separately as an enhancer.

17. The method of claim 7 wherein the drug formulation contains pain-relieving compounds or active pharmaceutical ingredients, their pharmaceutically acceptable salts or prodrugs thereof.

18. The method of claim 17, wherein the drug formulation contains compounds selected from the group consisting of preservatives, stabilizers, creams, gels, and isotonic solvents.

19. The method of claim 7 further comprising providing a TENS unit wherein the magnetic arrays and the TENS unit accelerate the diffusion of formulation dermally.

20. A method for administering drugs through a dermal layer comprising the following steps:
providing a magnetic device of at least on magnet to provide an effective magnetic sphere of influence of the magnetic field at the desired site of action;
providing a tissue permeable drug formulation which is positioned on the site of action;
positioning the magnetic device adjacent to tissue permeable drug formulation
wherein the magnetic sphere moves the drug through the dermal layer by interaction with the magnetic moment (induced or permanent) of the drug; and
providing a magnetic field for relief of pain.

21. A apparatus for administering a tissue permeable drug formulation at a predetermined site and predetermined rate through a dermal or mucosal layer of a mammal to a predetermined depth comprising:
a magnetic device of at least two magnets to provide an effective magnetic field gradient at the drug administration site to move molecules of the drug laterally over the distance between the administrative site and predetermined depth;
a delivery substrate into which the drug formulation is disposed; said substrate being positioned on the dermal or mucosal layer at the predetermined site between the magnetic device and the predetermined site;
whereby the magnetic field induces accelerated movement of the drug through the dermal or mucosal layer by interaction with the magnetic moment on the drug molecule around the dermal or mucosal layer cells in the magnetic field thereby accelerating the movement of the drug over that naturally provided by diffusion.

22. The apparatus of claim 21 wherein said magnetic device creates a field of at least 1.5 mT at a distance under the dermal of the desired depth of penetration of the drug.

23. The apparatus of claim 21 wherein the magnetic device is a permanent magnet.

24. The apparatus of claim 21 wherein the magnetic device is an electromagnet.

25. The apparatus of claim 21 wherein the apparatus includes a magnetic device supplying a magnetic field and an iontophoretic device supplying an electrical field.

26. The apparatus of claim 25 wherein the iontophoretic device is a TENS device.

27. The apparatus of claim 21 wherein the magnetic device includes an array of four magnets.

28. The apparatus of claim 27 wherein four magnets are arranged in alternating polarity on an iron flux ring.

29. The apparatus of claim 27 wherein each of the magnets has a residual induction value of at least about 1,400 mT.

30. The apparatus of claim 27 wherein each of the magnets has a diameter of about 12.7 mm and a thickness of about 6.35 mm.

31. The apparatus of claim 27 wherein the magnetic field at the center of the North Pole of the magnets is about 530 mT.

32. The apparatus of claim 27 wherein the magnetic field gradient at the site of action of adjacent poles of at least 1.5 mT/mm in the direction perpendicular to the axis from the magnetic poles.

33. The apparatus of claim 32 wherein the magnetic field gradient at the site of action of adjacent poles is at least about 1.5 mT/mm at a distance of 15 mm from the magnetic array.

34. A method for administering drugs through a mucous membrane comprising the following steps:
providing a magnetic device of at least one magnet to provide an effective magnetic sphere of influence at the desired site of action.
providing a tissue permeable drug formulation which is positioned on the site of action; and
positioning the magnetic device adjacent to said tissue permeable drug formulation
wherein the magnetic sphere of influence of the magnetic field moves the drug though the mucosal layer by interaction with the magnetic moment of the drug sufficient to increase the movement of the drug beyond the movement of diffusion alone.

35. A method for administering drugs through a mucous membrane, comprising the following steps:
providing a magnetic device in a magnet array of alternating polarity in which the magnetic poles are separated by a predetermined distance to provide an effective magnetic field creating a sphere of influence at the desired site of action from the magnetic fields of all adjacent poles;
providing a tissue permeable drug formulation which is positioned on the site of action; and
positioning the magnetic device adjacent to tissue permeable drug formulation
wherein the magnetic sphere of influence of the magnetic field moves the drug through the mucosal layer.

36. A method for administering drugs through a dermal or mucosal layer comprising the following steps:
providing a magnetic device of at least one magnet to provide an effective magnetic sphere of influence of the magnetic field at the desired site of action;
providing a tissue permeable drug formulation which is positioned on the site of action;
positioning the magnetic device adjacent to tissue permeable drug formulation
wherein the magnetic sphere moves the drug through the dermal or mucosal layer by interaction with the magnetic moment (induced or permanent) of the drug; and
providing a magnetic field for relief of pain.

37. A apparatus for administering drugs at a predetermined site through a dermal or mucosal layer of a mammal comprising: to a predetermined depth:
a magnetic device of at least one magnet to provide an effective magnetic field at the drug administration site;
a tissue permeable drug formulation;
a delivery substrate into which the drug formulation is disposed; said substrate being positionable on the dermal or mucosal layer at the predetermined site;
said magnetic device positioned on said substrate distal from the dermal or mucosal layer;
whereby the magnetic field induces movement of the drug through the dermal or mucosal layer by interaction with the magnetic moment thereby providing a magnetic field for relief of pain.
